SEQUENCE LISTING (28 sequences)

SEQ ID NO: 1 - protein, 28 aa, Homo sapiens
HGEGTFTSDVSSYLEEQAAKEFIAWLVK

SEQ ID NO: 2 - protein, 585 aa, Homo sapiens [sequence omitted]

SEQ ID NO: 3 - protein, 627 aa, Artificial
  GLP-1@`KFNoHZO500W5D01;yKaPrAP
HGEGTFTSDVSSYLEEQAAKEFIAWLVKGGGSSPPPGGGGSSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 4 - protein, 632 aa, Artificial
  GLP-1@`KFNoHZO500W5D01;yKaPrAP
HGEGTFTSDVSSYLEEQAAKEFIAWLVKGGGAPPPPPPPPPPSSGGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - protein, 639 aa, Artificial
  GLP-1@`KFNoHZO500W5D01;yKaPrAP
HGEGTFTSDVSSYLEEQAAKEFIAWLVKGGGSSGAPPPSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - DNA, 84 bp, Homo sapiens
cacggcgaagggacctttaccagtgatgtaagttcttatttggaagagcaagctgccaaggaattcattgcttggctggtgaaa

SEQ ID NO: 7 - DNA, 1758 bp, Homo sapiens
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 8 - DNA, 1884 bp, Artificial
  GLP-1@`KFNoHZO500WK\UKa1`BkPrAP
cacggcgaagggacctttaccagtgatgtaagttcttatttggaagagcaagctgccaaggaattcattgcttggctggtgaaaggtggtggatcttctccaccaccaggtggtggaggctcttcagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 9 - DNA, 1899 bp, Artificial
  GLP-1@`KFNoHZO500WK\UKa1`BkPrAP
cacggcgaagggacctttaccagtgatgtaagttcttatttggaagagcaagctgccaaggaattcattgcttggctggtgaaaggcgggggtgctccaccaccaccaccaccaccaccaccaccatcttccggaggcggtgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 10 - DNA, 1920 bp, Artificial
  GLP-1@`KFNoHZO500WK\UKa1`BkPrAP
cacggcgaagggacctttaccagtgatgtaagttcttatttggaagagcaagctgccaaggaattcattgcttggctggtgaaaggcggtggatcttctggtgctccaccaccatctggtggtggaggctctggaggtggaggttccggaggcgggggttcagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 11 - protein, 14 aa, Artificial
  A,=SkD
GGGSSPPPGGGGSS

SEQ ID NO: 12 - protein, 24 aa, Artificial
  A,=SkD
GGGSSGGGSSPPPAGGGSSGGGSS

SEQ ID NO: 13 - protein, 19 aa, Artificial
  A,=SkD
GGGAPPPPPPPPPPSSGGG

SEQ ID NO: 14 - protein, 24 aa, Artificial
  A,=SkD
AGGGAAGGGSSGGGPPPPPGGGGS

SEQ ID NO: 15 - protein, 15 aa, Artificial
  A,=SkD
GGSSGAPPPPGGGGS

SEQ ID NO: 16 - protein, 26 aa, Artificial
  A,=SkD
GGGSSGAPPPSGGGGSGGGGSGGGGS

SEQ ID NO: 17 - DNA, 187 bp, Artificial
  3 d6K4xHSAHZOF,6N5D(GLP-1@`KFNo)2;yRrF,6N
cacggcgaagggacctttaccagtgatgtaagttcttatttggaagagcaagctgccaaggaattcattgcttggctggtgaaacacggcgaagggacctttaccagtgatgtaagttcttatttggaagagcaagctgccaaggaattcattgcttggctggtgaaagatgcacacaagagtgagg

SEQ ID NO: 18 - DNA, 148 bp, Artificial
  3 d6K4xHSAHZOF,6N5DGLP-1@`KFNo-( Gly4Ser) 3;yRrF,6N
cacggcgaagggacctttaccagtgatgtaagttcttatttggaagagcaagctgccaaggaattcattgcttggctggtgaaaggtggtggaggctctggaggtggaggttccggaggcgggggttcagatgcacacaagagtgagg

SEQ ID NO: 19 - DNA, 163 bp, Artificial
  3 d6K4xHSAHZOF,6N5DGLP-1@`KFNo-( Gly4Ser) 4;yRrF,6N
cacggcgaagggacctttaccagtgatgtaagttcttatttggaagagcaagctgccaaggaattcattgcttggctggtgaaaggtggtggaggctctggtggtggaggctctggaggtggaggttccggaggcgggggttcagatgcacacaagagtgagg

SEQ ID NO: 20 - DNA, 145 bp, Artificial
  3 d6K4xHSAHZOF,6N5DGLP-1@`KFNo-E1;yRrF,6N
cacggcgaagggacctttaccagtgatgtaagttcttatttggaagagcaagctgccaaggaattcattgcttggctggtgaaaggtggtggatcttctccaccaccaggtggtggaggctcttcagatgcacacaagagtgagg

SEQ ID NO: 21 - DNA, 175 bp, Artificial
  3 d6K4xHSAHZOF,6N5DGLP-1@`KFNo-E2;yRrF,6N
cacggcgaagggacctttaccagtgatgtaagttcttatttggaagagcaagctgccaaggaattcattgcttggctggtgaaaggtggaggctcttcaggtggaggctcttcaccaccaccagctggtggaggctcttcaggtggaggctcttcagatgcacacaagagtgagg

SEQ ID NO: 22 - DNA, 160 bp, Artificial
  3 d6K4xHSAHZOF,6N5DGLP-1@`KFNo-E3;yRrF,6N
cacggcgaagggacctttaccagtgatgtaagttcttatttggaagagcaagctgccaaggaattcattgcttggctggtgaaaggcgggggtgctccaccaccaccaccaccaccaccaccaccatcttccggaggcggtgatgcacacaagagtgagg

SEQ ID NO: 23 - DNA, 175 bp, Artificial
  3 d6K4xHSAHZOF,6N5DGLP-1@`KFNo-E4;yRrF,6N
cacggcgaagggacctttaccagtgatgtaagttcttatttggaagagcaagctgccaaggaattcattgcttggctggtgaaagctggcgggggtgctgctggaggcgggtcttctggcgggggtccaccaccaccaccaggaggcgggggttcagatgcacacaagagtgagg

SEQ ID NO: 24 - DNA, 148 bp, Artificial
  3 d6K4xHSAHZOF,6N5DGLP-1@`KFNo-E5;yRrF,6N
cacggcgaagggacctttaccagtgatgtaagttcttatttggaagagcaagctgccaaggaattcattgcttggctggtgaaaggtggatcttctggtgctccaccaccaccaggaggcgggggttcagatgcacacaagagtgagg

SEQ ID NO: 25 - DNA, 181 bp, Artificial
  3 d6K4xHSAHZOF,6N5DGLP-1@`KFNo-E6;yRrF,6N
cacggcgaagggacctttaccagtgatgtaagttcttatttggaagagcaagctgccaaggaattcattgcttggctggtgaaaggcggtggatcttctggtgctccaccaccatctggtggtggaggctctggaggtggaggttccggaggcgggggttcagatgcacacaagagtgagg

SEQ ID NO: 26 - DNA, 40 bp, Artificial
  R{No
tctctcgagaaaagacacggcgaagggacctttaccagtg

SEQ ID NO: 27 - DNA, 19 bp, Artificial
  R}No
gatgcacacaagagtgagg

SEQ ID NO: 28 - DNA, 32 bp, Artificial
  R}No
ttagcggccgcttataagcctaaggcagcttg

(19) United States
(12) Patent Application Publication
(10) Pub. No.: US 2016/0280762 A1
(43) Pub. Date: Sep. 29, 2016

(54) GLP-1 ANALOG FUSION PROTEIN AND PREPARATION METHOD AND USE THEREOF

(71) Applicant: JIANGSU T-MAB BIOPHARMA CO., LTD, Jiangsu (CN)

(72) Inventors: YANSHAN HUANG, TAIZHOU, JIANGSU (CN); ZHIYU YANG, TAIZHOU, JIANGSU (CN); ZHENGXUE XU, TAIZHOU, JIANGSU (CN); JIWAN QIU, TAIZHOU, JIANGSU (CN)

(73) Assignee: JIANGSU T-MAB BIOPHARMA CO., LTD, TAIZHOU, JIANGSU (CN)

(21) Appl. No.: 14/909,143

(22) PCT Filed: Jul. 23, 2014
(86) PCT No.: PCT/CN2014/082798

§ 371 Date: Apr. 5, 2016

(30) Foreign Application Priority Data
Aug. 1, 2013 (CN) 201310331182.6

(51) Int. Cl. C07K 14/765; C12P 21/02; C07K 14/605

(52) U.S. Cl. CPC C07K 14/765; C07K 14/605; C12P 21/02; C07K 2319/31; A61K 38/00

(57) ABSTRACT

The present invention provides a novel GLP-1 analogue fusion protein and a method for preparing the fusion protein. The fusion protein consists of three regions as follows: GLP-1 analogue-linker peptide-HSA (Human Serum Albumin). Compounds which contain GLP-1 analogues prepared by adopting the present invention have the advantages of very low production cost, higher biological activity and better in-vivo and in-vitro stability. The fusion protein can be used for treating diabetes, obesity, irritable bowel syndrome and other diseases which can be benefited by reducing plasma glucose, inhibiting stomach and/or intestine movement and inhibiting stomach and/or intestine emptying or inhibiting food intake.

### FIELD OF THE INVENTION

The present invention relates to a novel GLP-1 analogue fusion protein and a method for preparing the fusion protein. The GLP-1 analogue fusion protein is used for treating diabetes and various related diseases or dysfunctions.

### BACKGROUND OF THE INVENTION

Glucagon-like peptide-1 (GLP-1) and analogues thereof such as Exendin-4 are widely used for researches on treating type-2 diabetes. Since GLP-1 polypeptides are quickly inactivated in vivo by protease dipeptidyl peptidase IV (DPP-IV) and the half-life period of GLP-1 polypeptides in plasma is very short, the widespread clinical application of GLP-1 polypeptides is difficult. Since Exendin-4 is not sensitive to enzymatic degradation of DPP-IV, the stability thereof is increased, however the molecular weight is lower (4187.61D) and the in-vivo half-life period is short, two times of injection are needed every day such that the clinical use is obstructed. At present, lots of efforts are made to solve the technical problem by means such as sustained release microsphere, PEG modification, fatty acid chain modification and albumin fusion, wherein the albumin fusion technique maintains biological and curative functions of target proteins and simultaneously greatly improves the in-vivo half-life period thereof through fusion with human albumin.

Although GLP-1 preparations and derivatives thereof are realistically feasible for treating diabetes, long-term continuous administration is needed once diabetic patients are diagnosed, the diabetic patients need to accept treatment throughout the entire life and thereby the requirements on the safety, economy and use convenience of the preparations are extremely high. However, the existing GLP-1/HSA fusion preparations have very great defects.

Firstly, compared with GLP-1 molecules, the molecular weight of albumin is huge. Therefore, after the fusion of them, due to steric hindrance, GLP-1/HSA fusion proteins substantially do not have biological activity. Albugon is a new GLP-1/HSA fusion protein designed by Laurie L. Baggio, et al., which is characterized in that an additional GLP-1 molecule is inserted therebetween as a spacer. However, about only 1% of biological activity thereof is reserved. The decrease of the biological activity causes the great increase of clinical dosage (Laurie L. Baggio, Qingling Huang, Theodore J. Brown, and Daniel J. Drucker, DIABETES Vol. 53: 2492-2500 (2004)). For example, with respect to a GLP-1 analogue Byetta®, the clinical administration dosage is only 5-10 μg per time and 1-2 times per day. However, the clinical effective administration dosage of Albugon reaches 4 mg per day, the mole number of which is increased by approximate 22 times. The great increase of clinical dosage causes two problems as follows: 1) potential immunogenicity risks are increased; the increase of dosage inevitably causes the increase of concentration of medicine preparations due to a limitation of administration volume, for example, the single-time dosage of the GLP-1 analogue preparation Byetta is only 5-10 μg (50 μl), the concentration is only 0.25 mg/ml, however the clinical single-time dosage of Albugon reaches 30 mg/person and the preparation concentration reaches up to 30-50 mg/ml; during transportation and storage of high-concentration protein preparations, the content of protein polymers are easily increased; researches have shown that the increase of treatment protein polymers will increase immunogenicity (Anne S. De Groot and David W. Scott, Trends Immunol Vol. 28 No. 11:482-490); recombined protein polymers will activate B-cell hyperplasia by cross-linking B-cell receptors such that B-cell and T-cell immunity is enabled (Rosenberg, A. S. Effects of protein aggregates: an immunologic perspective. AAPS J. 8: 501-507 (2006)); in addition, the recombined protein polymers are easily phagocytized by antigen presenting cells (APCs) such that the maturity of dendritic cells (DCs) is accelerated and thereby various immune responses are stimulated (Anne S. De Groot and David W. Scott, Trends Immunol Vol. 28 No. 11:482-490); and therefore, the remarkable increase of the dosage of the GLP-1/HSA fusion protein preparations will inevitably cause the increase of the risk of antibody production; and 2) the GLP-1/HSA fusion protein preparations need to be prepared by using extremely complex bioengineering technologies, the cost per unit quantity of protein is high and the great increase of the administration dosage will cause that the diabetic patients cannot afford the medicine.

Secondly, since most GLP-1 sequences are irregular and curly and are easily degraded due to attack by protease, an additional added second GLP-1 causes that Albugon is more easily attacked by protease and become instable. The instability shows defects in two aspects as follows: 1) when Albugon is recombined and expressed, regardless of a low-cost yeast expression system or a high-cost mammalian cell expression system, the GLP-1/HSA fusion protein secreted in culture supernatant is easily degraded by protease, and the degration not only lead to the decrease of the expression level, but also lead to the production of lots of non-uniform enzymatic hydrolysates, such that the final products are caused to be not uniform; and 2) after Albugon is injected in vivo, Albugon is easily degraded by protease and becomes ineffective during in-vivo circulation.

In addition, due to a limitation of product stability, at present all such products need to be stored and transported at low temperature and thereby the products are extremely inconvenient to carry by diabetic patients during outgoing and traveling.

### SUMMARY OF THE INVENTION

The purpose of the present invention is to overcome the defects in the prior art, design and prepare a novel GLP-1 analogue fusion protein, which consists of three regions as follows: GLP-1 analogue-linker peptide-HSA (Human Serum Albumin). Compared with the existing products, the remarkable advantages of this fusion protein are as following:

1. The thermal stability is better, the fusion protein can be stored for a long term at room temperature without causing the activity to be decreased, and the fusion protein can be conveniently carried with and used by patients.

2. The protease-resistant stability is better, the stability in fermented supernatant and in vivo is more than 3 times of that of the existing fusion protein and the industrial preparation is facilitated.

3. The biological activity is higher and the biological activity thereof is more than 10 times of that of the existing fusion protein.

Compounds which contain GLP-1 analogues prepared by adopting the present invention have the advantages of very low production cost, higher biological activity and better in-vivo and in-vitro stability, and thereby the compounds are expected to become a kind of better diabetes treatment medicines.

In a first aspect, the present invention discloses a novel GLP-1 analogue fusion protein, a structure of which is GLP-1 analogue-linker peptide-human serum albumin (HSA).

In the GLP-1 analogue fusion protein disclosed by the present invention, the first region in the structure thereof is a GLP-1 analogue, wherein a sequence thereof is as shown by SEQ ID NO. 1: HGEGTFTSDVSSYLEEQAAKEFIAWLVK, or at least maintains 85%, 90%, 95% or 99% of homology with SEQ ID NO. 1; further, the GLP-1 analogue can also comprises 2 or 3 repetitive sequences of GLP-1 or analogues thereof; and further, the first region can also be a homolog Exendin-4 with similar functions to GLP-1.

After natural GLP-1 is processed in vivo, first 6 amino acids of a mature peptide molecule are cut off. Therefore, according to a habit in the art, a first amino acid of GLP-1 is designated as No. 7. As shown in SEQ ID NO. 1, all amino acids in the polypeptide are continuously numbered. For example, a 7th site is a histidine and an 8th site is a glycine. Non-conservative positions in the GLP-1 sequence can be replaced by other amino acids without changing the activity thereof. For example, Gly8→Ala, Ser or Cys, Glu9→Asp, Gly, Ser, Cys, Thr, Asp, Gln, Tyr, Ala, Val, Ile, Leu, Met or Phe; Gly10→Ser, Cys, Thr, Asp, Glu, Tyr, Ala, Val, Ile, Leu, Met or Phe, Asp15→Glu, Val16→Leu or Tyr; Ser18→Lys, Glu21→Asp, Gly22→Glu or Ser; Glu23→Arg; Ala24→Arg; Lys26→Gly, Ser, Cys, Thr, Asp, Glu, Tyr, Ala, Val, Ile, Leu, Met, Phe, Arg; Lys34→Gly, Ser, Cys, Thr, Asp, Glu, Tyr, Ala, Val, Ile, Leu, Met, Phe, Arg; Arg36→Gly, Ser, Cys, Thr, Asp, Glu, Tyr, Ala, Val, Ile, Leu, Met, Phe, lys. C-terminal of GLP-1 can be in a deficiency of 1, 2 or 3 amino acids (Wolfgang Glaesner et al., U.S. Pat. No. 7,452,966). In the GLP-1 analogue fusion protein, the second region in the structure thereof is a connecting peptide with length which does not exceed 26 amino acids and a general formula is (Xaa)x-(Pro)y-(Xaa)z, wherein Xaa is one or any combination of a plurality of G, A and S, x, y and z are integers, x, z≧3, 26≧x+y+z≧14, 10≧y≧3, and 1≧y/(x+z)≧0.13. An N-terminal of the linker peptide is connected with a C-terminal of the first region through a peptide bond, and a C-terminal of the linker peptide is connected with an N-terminal of the HSA through a peptide bond.

That Xaa is one or any combination of a plurality of G, A and S refers to that Xaa at different positions can be freely selected from amino acid residues of G, A and S, and Xaa at different positions can be consistent and can also be inconsistent.

Further, the sequence of the linker peptide is selected from:

5 a)
(SEQ ID NO. 11)
GGGSSPPPGGGGSS
6 b)
(SEQ ID NO. 12)
GGGSSGGGSSPPPAGGGSSGGGSS
7 c)
(SEQ ID NO. 13)
GGGAPPPPPPPPPPSSGGG
8 d)
(SEQ ID NO. 14)
AGGGAAGGGSSGGGPPPPPGGGGS
9 e)
(SEQ ID NO. 15)
GGSSGAPPPPGGGGS
10 f)
(SEQ ID NO. 16)
GGGSSGAPPPSGGGGSGGGGSGGGGS

In the GLP-1 analogue fusion protein, a third region in the structure thereof is human serum albumin (HSA). A sequence thereof is as shown by SEQ ID NO. 2 or at least maintains 85%, 90%, 95% or 99% of homology with SEQ ID NO. 2. Non-conservative positions in the HSA sequence can be replaced by other amino acids without changing the activity thereof, such as Cys34→Ser, Leu407→Ala, Leu408→Val, Arg408→Val, Val409→Ala, Arg410→Ala, Lys413→Gln, Arg410→Ala (Plumridge et al., International Patent WO2011051489).

17 SEQ ID NO. 2:
18
DAHKSEVAHRFKDLGEENFKALVLIAFAQYLQQCPFEDHVKLVNEVTEFA
KTCVADE
19
SAENCDKSLHTLFGDKLCTVATLRETYGEMADCCAKQEPERNECFLQHKD
DNPNLP
20
RLVRPEVDVMCTAFHDNEETFLKKYLYEIARRHPYFYAPELLFFAKRYKA
AFTECCQ
21
AADKAACLLPKLDELRDEGKASSAKQRLKCASLQKFGERAFKAWAVARLS
QRFPKA
22
EFAEVSKLVTDLTKVHTECCHGDLLECADDRADLAKYICENQDSISSKLK
ECCEKPLL
23
EKSHCIAEVENDEMPADLPSLAADFVESKDVCKNYAEAKDVFLGMFLYEY
ARRHPD
24
YSVVLLLRLAKTYETTLEKCCAAADPHECYAKVFDEFKPLVEEPQNLIKQ
NCELFEQ
25
LGEYKFQNALLVRYTKKVPQVSTPTLVEVSRNLGKVGSKCCKHPEAKRMP
CAEDYL
26
SVVLNQLCVLHEKTPVSDRVTKCCTESLVNRRPCFSALEVDETYVPKEFN
AETFTFH
27
ADICTLSEKERQIKKQTALVELVKHKPKATKEQLKAVMDDFAAFVEKCCK
ADDKETC
28
FAEEGKKLVAASQAALGL

In preferred embodiments of the present invention, an amino acid sequence of the GLP-1 analogue fusion protein is selected from SEQ ID NO. 3-5.

a) SEQ ID NO. 3:
HGEGTFTSDVSSYLEEQAAKEFIAWLVKGGGSSPPPGGGGSSDAHKSEVA
HRFKDLGEENFKALVLIAFAQYLQQCPFEDHVKLVNEVTEFAKTCVADES
AENCDKSLHTLFGDKLCTVATLRETYGEMADCCAKQEPERNECFLQHKDD
NPNLPRLVRPEVDVMCTAFHDNEETFLKKYLYEIARRHPYFYAPELLFFA
KRYKAAFTECCQAADKAACLLPKLDELRDEGKASSAKQRLKCASLQKFGE
RAFKAWAVARLSQRFPKAEFAEVSKLVTDLTKVHTECCHGDLLECADDRA
DLAKYICENQDSISSKLKECCEKPLLEKSHCIAEVENDEMPADLPSLAAD
FVESKDVCKNYAEAKDVFLGMFLYEYARRHPDYSVVLLLRLAKTYETTLE
KCCAAADPHECYAKVFDEFKPLVEEPQNLIKQNCELFEQLGEYKFQNALL
VRYTKKVPQVSTPTLVEVSRNLGKVGSKCCKHPEAKRMPCAEDYLSVVLN
QLCVLHEKTPVSDRVTKCCTESLVNRRPCFSALEVDETYVPKEFNAETFT
FHADICTLSEKERQIKKQTALVELVKHKPKATKEQLKAVMDDFAAFVEKC
CKADDKETCFAEEGKKLVAASQAALGL
b)SEQ ID NO. 4:
HGEGTFTSDVSSYLEEQAAKEFIAWLVKGGGAPPPPPPPPPPSSGGGDAH
KSEVAHRFKDLGEENFKALVLIAFAQYLQQCPFEDHVKLVNEVTEFAKTC
VADESAENCDKSLHTLFGDKLCTVATLRETYGEMADCCAKQEPERNECFL
QHKDDNPNLPRLVRPEVDVMCTAFHDNEETFLKKYLYEIARRHPYFYAPE
LLFFAKRYKAAFTECCQAADKAACLLPKLDELRDEGKASSAKQRLKCASL
QKFGERAFKAWAVARLSQRFPKAEFAEVSKLVTDLTKVHTECCHGDLLEC
ADDRADLAKYICENQDSISSKLKECCEKPLLEKSHCIAEVENDEMPADLP
SLAADFVESKDVCKNYAEAKDVFLGMFLYEYARRHPDYSVVLLLRLAKTY
ETTLEKCCAAADPHECYAKVFDEFKPLVEEPQNLIKQNCELFEQLGEYKF
QNALLVRYTKKVPQVSTPTLVEVSRNLGKVGSKCCKHPEAKRMPCAEDYL
SVVLNQLCVLHEKTPVSDRVTKCCTESLVNRRPCFSALEVDETYVPKEFN
AETFTFHADICTLSEKERQIKKQTALVELVKHKPKATKEQLKAVMDDFAA
FVEKCCKADDKETCFAEEGKKLVAASQAALGL
c) SEQ ID NO. 5:
HGEGTFTSDVSSYLEEQAAKEFIAWLVKGGGSSGAPPPSGGGGSGGGGSG
GGGSDAHKSEVAHRFKDLGEENFKALVLIAFAQYLQQCPFEDHVKLVNEV
TEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRH
PYFYAPELLFFAKRYKAAFTECCQAADKAACLLPKLDELRDEGKASSAKQ
RLKCASLQKFGERAFKAWAVARLSQRFPKAEFAEVSKLVTDLTKVHTECC
HGDLLECADDRADLAKYICENQDSISSKLKECCEKPLLEKSHCIAEVEND
EMPADLPSLAADFVESKDVCKNYAEAKDVFLGMFLYEYARRHPDYSVVLL
LRLAKTYETTLEKCCAAADPHECYAKVFDEFKPLVEEPQNLIKQNCELFE
QLGEYKFQNALLVRYTKKVPQVSTPTLVEVSRNLGKVGSKCCKHPEAKRM
PCAEDYLSVVLNQLCVLHEKTPVSDRVTKCCTESLVNRRPCFSALEVDET
YVPKEFNAETFTFHADICTLSEKERQIKKQTALVELVKHKPKATKEQLKA
VMDDFAAFVEKCCKADDKETCFAEEGKKLVAASQAALGL

In a second aspect, the present invention discloses a polynucleotide coding the GLP-1 analogue fusion protein.

In preferred embodiments of the present invention, a nucleotide coding sequence of the GLP-1 analogue fusion protein is SEQ ID NO. 10 and a corresponding protein sequence thereof is SEQ ID NO. 5. The nucleotide coding sequence of the GLP-1 analogue fusion protein disclosed by the present invention can also be SEQ ID NO. 8 and the corresponding protein sequence thereof is SEQ ID NO. 3; or the nucleotide coding sequence is SEQ ID NO. 9 and the corresponding protein sequence thereof is SEQ ID NO. 4.

SEQ ID NO. 8: nucleotide coding sequence of GLP-1
analogue fusion protein
CACGGCGAAGGGACCTTTACCAGTGATGTAAGTTCTTATTTGGAAGAGCA
AGCTGCCAAGGAATTCATTGCTTGGCTGGTGAAAGGTGGTGGATCTTCTC
CACCACCAGGTGGTGGAGGCTCTTCAGATGCACACAAGAGTGAGGTTGCT
CATCGGTTTAAAGATTTGGGAGAAGAAAATTTCAAAGCCTTGGTGTTGAT
TGCCTTTGCTCAGTATCTTCAGCAGTGTCCATTTGAAGATCATGTAAAAT
TAGTGAATGAAGTAACTGAATTTGCAAAAACATGTGTTGCTGATGAGTCA
GCTGAAAATTGTGACAAATCACTTCATACCCTTTTTGGAGACAAATTATG
CACAGTTGCAACTCTTCGTGAAACCTATGGTGAAATGGCTGACTGCTGTG
CAAAACAAGAACCTGAGAGAAATGAATGCTTCTTGCAACACAAAGATGAC
AACCCAAACCTCCCCCGATTGGTGAGACCAGAGGTTGATGTGATGTGCAC
TGCTTTTCATGACAATGAAGAGACATTTTTGAAAAAATACTTATATGAAA
TTGCCAGAAGACATCCTTACTTTTATGCCCCGGAACTCCTTTTCTTTGCT
AAAAGGTATAAAGCTGCTTTTACAGAATGTTGCCAAGCTGCTGATAAAGC
TGCCTGCCTGTTGCCAAAGCTCGATGAACTTCGGGATGAAGGGAAGGCTT
CGTCTGCCAAACAGAGACTCAAGTGTGCCAGTCTCCAAAAATTTGGAGAA
AGAGCTTTCAAAGCATGGGCAGTAGCTCGCCTGAGCCAGAGATTTCCCAA
AGCTGAGTTTGCAGAAGTTTCCAAGTTAGTGACAGATCTTACCAAAGTCC
ACACGGAATGCTGCCATGGAGATCTGCTTGAATGTGCTGATGACAGGGCG
GACCTTGCCAAGTATATCTGTGAAAATCAAGATTCGATCTCCAGTAAACT
GAAGGAATGCTGTGAAAAACCTCTGTTGGAAAAATCCCACTGCATTGCCG
AAGTGGAAAATGATGAGATGCCTGCTGACTTGCCTTCATTAGCTGCTGAT
TTTGTTGAAAGTAAGGATGTTTGCAAAAACTATGCTGAGGCAAAGGATGT
CTTCCTGGGCATGTTTTTGTATGAATATGCAAGAAGGCATCCTGATTACT
CTGTCGTGCTGCTGCTGAGACTTGCCAAGACATATGAAACCACTCTAGAG
AAGTGCTGTGCCGCTGCAGATCCTCATGAATGCTATGCCAAAGTGTTCGA
TGAATTTAAACCTCTTGTGGAAGAGCCTCAGAATTTAATCAAACAAAATT
GTGAGCTTTTTGAGCAGCTTGGAGAGTACAAATTCCAGAATGCGCTATTA
GTTCGTTACACCAAGAAAGTACCCCAAGTGTCAACTCCAACTCTTGTAGA
GGTCTCAAGAAACCTAGGAAAAGTGGGCAGCAAATGTTGTAAACATCCTG
AAGCAAAAAGAATGCCCTGTGCAGAAGACTATCTATCCGTGGTCCTGAAC
CAGTTATGTGTGTTGCATGAGAAAACGCCAGTAAGTGACAGAGTCACAAA
ATGCTGCACAGAGTCCTTGGTGAACAGGCGACCATGCTTTTCAGCTCTGG
AAGTCGATGAAACATACGTTCCCAAAGAGTTTAATGCTGAAACATTCACC
TTCCATGCAGATATATGCACACTTTCTGAGAAGGAGAGACAAATCAAGAA
ACAAACTGCACTTGTTGAGCTTGTGAAACACAAGCCCAAGGCAACAAAAG
AGCAACTGAAAGCTGTTATGGATGATTTCGCAGCTTTTGTAGAGAAGTGC
TGCAAGGCTGACGATAAGGAGACCTGCTTTGCCGAGGAGGGTAAAAAACT
TGTTGCTGCAAGTCAAGCTGCCTTAGGCTTATAA
SEQ ID NO. 9: nucleotide coding sequence of GLP-1
analogue fusion protein
CACGGCGAAGGGACCTTTACCAGTGATGTAAGTTCTTATTTGGAAGAGCA
AGCTGCCAAGGAATTCATTGCTTGGCTGGTGAAAGGCGGGGGTGCTCCAC
CACCACCACCACCACCACCACCACCATCTTCCGGAGGCGGTGATGCACAC
AAGAGTGAGGTTGCTCATCGGTTTAAAGATTTGGGAGAAGAAAATTTCAA
AGCCTTGGTGTTGATTGCCTTTGCTCAGTATCTTCAGCAGTGTCCATTTG
AAGATCATGTAAAATTAGTGAATGAAGTAACTGAATTTGCAAAAACATGT
GTTGCTGATGAGTCAGCTGAAAATTGTGACAAATCACTTCATACCCTTTT
TGGAGACAAATTATGCACAGTTGCAACTCTTCGTGAAACCTATGGTGAAA
TGGCTGACTGCTGTGCAAAACAAGAACCTGAGAGAAATGAATGCTTCTTG
CAACACAAAGATGACAACCCAAACCTCCCCCGATTGGTGAGACCAGAGGT
TGATGTGATGTGCACTGCTTTTCATGACAATGAAGAGACATTTTTGAAAA
AATACTTATATGAAATTGCCAGAAGACATCCTTACTTTTATGCCCCGGAA
CTCCTTTTCTTTGCTAAAAGGTATAAAGCTGCTTTTACAGAATGTTGCCA
AGCTGCTGATAAAGCTGCCTGCCTGTTGCCAAAGCTCGATGAACTTCGGG
ATGAAGGGAAGGCTTCGTCTGCCAAACAGAGACTCAAGTGTGCCAGTCTC
CAAAAATTTGGAGAAAGAGCTTTCAAAGCATGGGCAGTAGCTCGCCTGAG
CCAGAGATTTCCCAAAGCTGAGTTTGCAGAAGTTTCCAAGTTAGTGACAG
ATCTTACCAAAGTCCACACGGAATGCTGCCATGGAGATCTGCTTGAATGT
GCTGATGACAGGGCGGACCTTGCCAAGTATATCTGTGAAAATCAAGATTC
GATCTCCAGTAAACTGAAGGAATGCTGTGAAAAACCTCTGTTGGAAAAAT
CCCACTGCATTGCCGAAGTGGAAAATGATGAGATGCCTGCTGACTTGCCT
TCATTAGCTGCTGATTTTGTTGAAAGTAAGGATGTTTGCAAAAACTATGC
TGAGGCAAAGGATGTCTTCCTGGGCATGTTTTTGTATGAATATGCAAGAA
GGCATCCTGATTACTCTGTCGTGCTGCTGCTGAGACTTGCCAAGACATAT
GAAACCACTCTAGAGAAGTGCTGTGCCGCTGCAGATCCTCATGAATGCTA
TGCCAAAGTGTTCGATGAATTTAAACCTCTTGTGGAAGAGCCTCAGAATT
TAATCAAACAAAATTGTGAGCTTTTTGAGCAGCTTGGAGAGTACAAATTC
CAGAATGCGCTATTAGTTCGTTACACCAAGAAAGTACCCCAAGTGTCAAC
TCCAACTCTTGTAGAGGTCTCAAGAAACCTAGGAAAAGTGGGCAGCAAAT
GTTGTAAACATCCTGAAGCAAAAAGAATGCCCTGTGCAGAAGACTATCTA
TCCGTGGTCCTGAACCAGTTATGTGTGTTGCATGAGAAAACGCCAGTAAG
TGACAGAGTCACAAAATGCTGCACAGAGTCCTTGGTGAACAGGCGACCAT
GCTTTTCAGCTCTGGAAGTCGATGAAACATACGTTCCCAAAGAGTTTAAT
GCTGAAACATTCACCTTCCATGCAGATATATGCACACTTTCTGAGAAGGA
GAGACAAATCAAGAAACAAACTGCACTTGTTGAGCTTGTGAAACACAAGC
CCAAGGCAACAAAAGAGCAACTGAAAGCTGTTATGGATGATTTCGCAGCT
TTTGTAGAGAAGTGCTGCAAGGCTGACGATAAGGAGACCTGCTTTGCCGA
GGAGGGTAAAAAACTTGTTGCTGCAAGTCAAGCTGCCTTAGGCTTATAA
SEQ ID NO. 10: nucleotide coding sequence of GLP-1
analogue fusion protein
CACGGCGAAGGGACCTTTACCAGTGATGTAAGTTCTTATTTGGAAGAGCA
AGCTGCCAAGGAATTCATTGCTTGGCTGGTGAAAGGCGGTGGATCTTCTG
GTGCTCCACCACCATCTGGTGGTGGAGGCTCTGGAGGTGGAGGTTCCGGA
GGCGGGGGTTCAGATGCACACAAGAGTGAGGTTGCTCATCGGTTTAAAGA
TTTGGGAGAAGAAAATTTCAAAGCCTTGGTGTTGATTGCCTTTGCTCAGT
ATCTTCAGCAGTGTCCATTTGAAGATCATGTAAAATTAGTGAATGAAGTA
ACTGAATTTGCAAAAACATGTGTTGCTGATGAGTCAGCTGAAAATTGTGA
CAAATCACTTCATACCCTTTTTGGAGACAAATTATGCACAGTTGCAACTC
TTCGTGAAACCTATGGTGAAATGGCTGACTGCTGTGCAAAACAAGAACCT
GAGAGAAATGAATGCTTCTTGCAACACAAAGATGACAACCCAAACCTCCC
CCGATTGGTGAGACCAGAGGTTGATGTGATGTGCACTGCTTTTCATGACA
ATGAAGAGACATTTTTGAAAAAATACTTATATGAAATTGCCAGAAGACAT
CCTTACTTTTATGCCCCGGAACTCCTTTTCTTTGCTAAAAGGTATAAAGC
TGCTTTTACAGAATGTTGCCAAGCTGCTGATAAAGCTGCCTGCCTGTTGC
CAAAGCTCGATGAACTTCGGGATGAAGGGAAGGCTTCGTCTGCCAAACAG
AGACTCAAGTGTGCCAGTCTCCAAAAATTTGGAGAAAGAGCTTTCAAAGC
ATGGGCAGTAGCTCGCCTGAGCCAGAGATTTCCCAAAGCTGAGTTTGCAG
AAGTTTCCAAGTTAGTGACAGATCTTACCAAAGTCCACACGGAATGCTGC
CATGGAGATCTGCTTGAATGTGCTGATGACAGGGCGGACCTTGCCAAGTA
TATCTGTGAAAATCAAGATTCGATCTCCAGTAAACTGAAGGAATGCTGTG
AAAAACCTCTGTTGGAAAAATCCCACTGCATTGCCGAAGTGGAAAATGAT
GAGATGCCTGCTGACTTGCCTTCATTAGCTGCTGATTTTGTTGAAAGTAA
GGATGTTTGCAAAAACTATGCTGAGGCAAAGGATGTCTTCCTGGGCATGT
TTTTGTATGAATATGCAAGAAGGCATCCTGATTACTCTGTCGTGCTGCTG
CTGAGACTTGCCAAGACATATGAAACCACTCTAGAGAAGTGCTGTGCCGC
TGCAGATCCTCATGAATGCTATGCCAAAGTGTTCGATGAATTTAAACCTC
TTGTGGAAGAGCCTCAGAATTTAATCAAACAAAATTGTGAGCTTTTTGAG
CAGCTTGGAGAGTACAAATTCCAGAATGCGCTATTAGTTCGTTACACCAA
GAAAGTACCCCAAGTGTCAACTCCAACTCTTGTAGAGGTCTCAAGAAACC
TAGGAAAAGTGGGCAGCAAATGTTGTAAACATCCTGAAGCAAAAAGAATG
CCCTGTGCAGAAGACTATCTATCCGTGGTCCTGAACCAGTTATGTGTGTT
GCATGAGAAAACGCCAGTAAGTGACAGAGTCACAAAATGCTGCACAGAGT
CCTTGGTGAACAGGCGACCATGCTTTTCAGCTCTGGAAGTCGATGAAACA
TACGTTCCCAAAGAGTTTAATGCTGAAACATTCACCTTCCATGCAGATAT
ATGCACACTTTCTGAGAAGGAGAGACAAATCAAGAAACAAACTGCACTTG
TTGAGCTTGTGAAACACAAGCCCAAGGCAACAAAAGAGCAACTGAAAGCT
GTTATGGATGATTTCGCAGCTTTTGTAGAGAAGTGCTGCAAGGCTGACGA
TAAGGAGACCTGCTTTGCCGAGGAGGGTAAAAAACTTGTTGCTGCAAGTC
AAGCTGCCTTAGGCTTATAA

The nucleotide sequence coding the GLP-1 analogue fusion protein can be prepared through any proper techniques well-known by one skilled in the art, including, but not limited to, recombinant DNA technique, chemical synthesis and the like; and as well, firstly a nucleotide sequence having a GLP-1 amino acid sequence can be synthesized and then sequences are interposed, replaced and removed through site-directed mutation, directed mutagenesis or other techniques well-known in the art to obtain the needed nucleotide sequence.

The nucleotide sequence coding carrier protein can be prepared through any proper techniques well-known by one skilled in the art. In one specific embodiment of the present invention, the nucleotide sequence of the carrier protein is a nucleotide sequence coding HSA or at least maintains 95% of consistency with the nucleotide sequence coding HSA.

For a technique of fusion between the nucleotide sequence coding the GLP-1 analogue and nucleotide sequence coding the carrier protein, see general description in the art, such as Molecular Cloning (J. Sambrook et al., Science Press, 1995).

In a third aspect, the present invention discloses a method for preparing the foresaid fusion protein. The method comprises the following steps: constructing an expression vector containing a gene sequence of the fusion protein, then transforming the expression vector containing the gene sequence of the fusion protein to a host cell for induced expression, and separating and obtaining the fusion protein from expression products.

The expression vector for constructing the gene sequence containing the fusion protein can be obtained by firstly synthesizing the nucleotide sequence coding the GLP-1 analogue, then fusing the nucleotide sequence with the nucleotide sequence coding the HSA and finally constructing to a proper expression vector.

The gene sequence expressing the GLP-1 analogue fusion protein can be expressed through expression systems well-known by one skilled in the art, including, but not limited to, bacteria transformed by using vectors such as recombinant phages and plasmids, yeast transformed by using yeast expression vectors, filamentous fungi transformed by using fungus vectors, insect cells and animal cells infected by using virus vectors and the like. In one specific embodiment of the present invention, the expression system selects and uses Pichia pastoris secretion expression. Pichia pastoris is high in expression level and low in cost and has the advantages of protein processing, folding and posttranslational modification of a eukaryotic expression system. During actual production, cells can be cultured through a shake flask in a laboratory or can be cultured through fermentation in a fermentation tank (including continuous, batch-to-batch, fed-batch and solid state fermentation).

The fusion protein which is secreted into culture medium can be purified through methods well-known by one skilled in the art, including, but not limited to, ultrafiltration, ammonium sulfate precipitation, acetone precipitation, ion exchange chromatography, hydrophobic chromatography, reversed phase chromatography, molecular sieve chromatography and the like. In one specific embodiment of the present invention, the inventor adopts a three-step chromatographic means which joints affinity chromatography, hydrophobic chromatography and ion exchange chromatography to enable the fusion protein to be purified uniformly.

In a fourth aspect, the present invention discloses application of the GLP-1 analogue fusion protein to preparation of medicines for treating diabetes and related diseases.

In a fifth aspect, the present invention discloses a pharmaceutical composition containing the GLP-1 analogue fusion protein and at least one pharmaceutically acceptable carrier or excipient.

The pharmaceutical composition is mainly used for treating diabetes and related diseases. The related diseases include type-2 diabetes, type-1 diabetes, obesity, serious cardiovascular events of patients suffering from type-2 diabetes and other serious complications (Madsbad S, Kielgast U, Asmar M, et al. Diabetes Obes Metab. 2011 May; 13(5):394-407; Issa C M, Azar S T. Curr Diab Rep, 2012 October; 12(5):560-567; Neff L M, Kushner R F. Diabetes Metab Syndr Obes, 2010 Jul. 20; 3:263-273; Sivertsen J, Rosenmeier J, Holst J J, et al. Nat Rev Cardiol, 2012 Jan. 31; 9(4):209-222).

Indolent inorganic or organic carriers well-known by one skilled in the art include (but not limited to) saccharides and derivatives thereof, amino acids or derivatives thereof, surfactants, vegetable oil, wax, fat and polyhydroxy compounds such as polyethylene glycol, alcohols, glycerol, various preservatives, antioxidants, stabilizers, salts, buffer solution, water and the like can also be added therein, and these substances are used for improving the stability of the composition or improving the activity or biological effectiveness thereof according to the needs.

The pharmaceutical composition disclosed by the present invention can be prepared by adopting techniques well-known by one skilled in the art, including liquid or gel, freeze-drying or other forms, so as to produce medicines which are stable during storage and are suitable for administration to human or animals.

In a sixth aspect, the present invention discloses a method for treating diabetes and diabetes-related diseases, comprising the step of administrating the GLP-1 analogue fusion protein to an object.

For the method for treating patients suffering from non-insulin dependent or insulin dependent diabetic patients, obesity and various other diseases by using the foresaid fusion protein, a reference can be made to the existing GLP-1 medicine preparations such as Byetta® (GLP-1 analogue peptide), Albugon® (GLP-1/HSA fusion protein) and Dulaglutide® (GLP-1/Fc fusion protein, and the dosage range thereof is 0.05-1 mg/kg.

The protein disclosed by the present invention can be administrated solely, administrated by means of various combinations or administrated together with other treatment preparations.

In the present invention, the following abbreviations are used:

GLP-1 (glucagon like protein-1); HSA (human serum albumin)

### DESCRIPTION OF THE DRAWINGS

FIG. 1 illustrates an SDS-PAGEof the expression of GLP-1 analogue fusion proteins with different structures, wherein lanes 1-9 respectively are expression results of fusion proteins with sequences No. 1-9.

FIGS. 2A-D illustrate results of a pharmacodynamic test of a GLP-1 analogue fusion protein after single-dose subcutaneous injection to a normal rhesus monkey, wherein

FIG. 2A illustrates blood glucose levels of a rhesus monkey during graded glucose infusion after 1 day after subcutaneous injection of GLP-1-E3-HSA.

FIG. 2B illustrates blood glucose levels of a rhesus monkey during graded glucose infusion after 4 days after subcutaneous injection of GLP-1-E3-HSA.

FIG. 2C illustrates insulin levels of a rhesus monkey during graded glucose infusion after 1 day after subcutaneous injection of GLP-1-E3-HSA.

FIG. 2D illustrates insulin levels of a rhesus monkey during graded glucose infusion after 4 day after subcutaneous injection of GLP-1-E3-HSA.

FIG. 3 illustrates a concentration-time curve chart after single-dose administration to a rhesus monkey.

### DESCRIPTION OF SEQUENCES

SEQ ID NO. 1: amino acid sequence of GLP-1 analogue

SEQ ID NO. 2: amino acid sequence of HSA

SEQ ID NO. 3: amino acid sequence of GLP-1 analogue fusion protein

SEQ ID NO. 4: amino acid sequence of GLP-1 analogue fusion protein

SEQ ID NO. 5: amino acid sequence of GLP-1 analogue fusion protein

SEQ ID NO. 6: nucleotide coding sequence of GLP-1 analogue

SEQ ID NO. 7: nucleotide coding sequence of HSA

SEQ ID NO. 8: nucleotide coding sequence of GLP-1 analogue fusion protein

SEQ ID NO. 9: nucleotide coding sequence of GLP-1 analogue fusion protein

SEQ ID NO. 10: nucleotide coding sequence of GLP-1 analogue fusion protein

### DESCRIPTION OF THE EMBODIMENTS

The present invention will be described below through specific embodiments. One skilled in the art can easily understand other advantages and efficacies of the present invention according to the contents disclosed by the description. The present invention can also be implemented or applied through other different specific embodiments. Various modifications or changes can be made to all details in the description based on different points of view and applications without departing from the spirit of the present invention.

Unless otherwise stated, experiment methods, detection methods, preparation methods disclosed by the present invention adopt conventional molecular biology, biochemistry, chromatin structure and analysis, analytical chemistry, cell culture, recombinant DNA techniques in the art and conventional techniques in related arts.

### Embodiment 1: Construction of Recombinant Fusion Protein Expression Plasmid

Nucleotide coding sequence of GLP-1 analogue (SEQ ID NO. 6):

CACGGCGAAGGGACCTTTACCAGTGATGTAAGTTCTTATTTGGAAGAGCA
AGCTGCCAAGGAATTCATTGCTTGGCTGGTGAAA

1.1 (GLP-1 analogue)2 gene segment with an HSA fusion segment at 3′-terminal:

An oligonucleotide sequence (SEQ ID NO. 17) as follow was artificially synthesized:

CACGGCGAAGGGACCTTTACCAGTGATGTAAGTTCTTATTTGGAAGAGCA
AGCTGCCAAGGAATTCATTGCTTGGCTGGTGAAACACGGCGAAGGGACCT
TTACCAGTGATGTAAGTTCTTATTTGGAAGAGCAAGCTGCCAAGGAATTC
ATTGCTTGGCTGGTGAAAGATGCACACAAGAGTGAGG

wherein the single line marked part is a (GLP-1 analogue)2 gene sequence and the other part is an HSA N-terminal coding sequence.

1.2 GLP-1 analogue-(Gly4Ser)3 gene segment with an HSA fusion segment at 3′-terminal:

An oligonucleotide sequence (SEQ ID NO. 18) as follow was artificially synthesized:

CACGGCGAAGGGACCTTTACCAGTGATGTAAGTTCTTATTTGGAAGAGCA
AGCTGCCAAGGAATTCATTGCTTGGCTGGTGAAAGGTGGTGGAGGCTCTG
GAGGTGGAGGTTCCGGAGGCGGGGGTTCAGATGCACACAAGAGTGAGG

wherein the single line marked part is a GLP-1 analogue-(Gly₄Ser)₃ gene sequence and the other part is an HSA N-terminal coding sequence.

1.3 GLP-1 analogue-(Gly₄Ser)₄ gene segment with an HSA fusion segment at 3′-terminal:

An oligonucleotide sequence (SEQ ID NO. 19) as follow was artificially synthesized:

CACGGCGAAGGGACCTTTACCAGTGATGTAAGTTCTTATTTGGAAGAGCA
AGCTGCCAAGGAATTCATTGCTTGGCTGGTGAAAGGTGGTGGAGGCTCTG
GTGGTGGAGGCTCTGGAGGTGGAGGTTCCGGAGGCGGGGGTTCAGATGCA
CACAAGAGTGAGG

wherein the single line marked part is a GLP-1 analogue-(Gly₄Ser)₄ gene sequence and the other part is an HSA N-terminal coding sequence.

1.4 GLP-1 analogue-El gene segment with an HSA fusion segment at 3′-terminal:

An oligonucleotide sequence (SEQ ID NO. 20) as follow was artificially synthesized:

CACGGCGAAGGGACCTTTACCAGTGATGTAAGTTCTTATTTGGAAGAGCA
AGCTGCCAAGGAATTCATTGCTTGGCTGGTGAAAGGTGGTGGATCTTCTC
CACCACCAGGTGGTGGAGGCTCTTCAGATGCACACAAGAGTGAGG

wherein the single line marked part is a GLP-1 analogue-E1 gene sequence and the other part is an HSA N-terminal coding sequence.

1.5 GLP-1 analogue-E2 gene segment with an HSA fusion segment at 3′-terminal: An oligonucleotide sequence (SEQ ID NO. 21) as follow was artificially synthesized:

CACGGCGAAGGGACCTTTACCAGTGATGTAAGTTCTTATTTGGAAGAGCA
AGCTGCCAAGGAATTCATTGCTTGGCTGGTGAAAGGTGGAGGCTCTTCAG
GTGGAGGCTCTTCACCACCACCAGCTGGTGGAGGCTCTTCAGGTGGAGGC
TCTTCAGATGCACACAAGAGTGAGG

wherein the single line marked part is a GLP-1 analogue-E2 gene sequence and the other part is an HSA N-terminal coding sequence.

1.6 GLP-1 analogue-E3 gene segment with an HSA fusion segment at 3′-terminal:

An oligonucleotide sequence (SEQ ID NO. 22) as follow was artificially synthesized:

CACGGCGAAGGGACCTTTACCAGTGATGTAAGTTCTTATTTGGAAGAGCA
AGCTGCCAAGGAATTCATTGCTTGGCTGGTGAAAGGCGGGGGTGCTCCAC
CACCACCACCACCACCACCACCACCATCTTCCGGAGGCGGTGATGCACAC
AAGAGTGAGG

wherein the single line marked part is a GLP-1 analogue-E3 gene sequence and the other part is an HSA N-terminal coding sequence.

1.7 GLP-1 analogue-E4 gene segment with an HSA fusion segment at 3′-terminal:

An oligonucleotide sequence (SEQ ID NO. 23) as follow was artificially synthesized:

CACGGCGAAGGGACCTTTACCAGTGATGTAAGTTCTTATTTGGAAGAGCA
AGCTGCCAAGGAATTCATTGCTTGGCTGGTGAAAGCTGGCGGGGGTGCTG
CTGGAGGCGGGTCTTCTGGCGGGGGTCCACCACCACCACCAGGAGGCGGG
GGTTCAGATGCACACAAGAGTGAGG

wherein the single line marked part is a GLP-1 analogue-E4 gene sequence and the other part is an HSA N-terminal coding sequence.

1.8 GLP-1 analogue-ES gene segment with an HSA fusion segment at 3′-terminal:

An oligonucleotide sequence (SEQ ID NO. 24) as follow was artificially synthesized:

CACGGCGAAGGGACCTTTACCAGTGATGTAAGTTCTTATTTGGAAGAGCA
AGCTGCCAAGGAATTCATTGCTTGGCTGGTGAAAGGTGGATCTTCTGGTG
CTCCACCACCACCAGGAGGCGGGGGTTCAGATGCACACAAGAGTGAGG

wherein the single line marked part is a GLP-1 analogue-E5 gene sequence and the other part is an HSA N-terminal coding sequence.

1.9 GLP-1 analogue-E6 gene segment with an HSA fusion segment at 3′-terminal:

An oligonucleotide sequence (SEQ ID NO. 25) as follow was artificially synthesized:

CACGGCGAAGGGACCTTTACCAGTGATGTAAGTTCTTATTTGGAAGAGCA
AGCTGCCAAGGAATTCATTGCTTGGCTGGTGAAAGGCGGTGGATCTTCTG
GTGCTCCACCACCATCTGGTGGTGGAGGCTCTGGAGGTGGAGGTTCCGGA
GGCGGGGGTTCAGATGCACACAAGAGTGAGG

wherein the single line marked part is a GLP-1 analogue-E6 gene sequence and the other part is an HSA N-terminal coding sequence.

(SEQ ID NO. 11)
E1: GGGSSPPPGGGGSS
(SEQ ID NO. 12)
E2: GGGSSGGGSSPPPAGGGSSGGGSS
(SEQ ID NO. 13)
E3: GGGAPPPPPPPPPPSSGGG
(SEQ ID NO. 14)
E4: AGGGAAGGGSSGGGPPPPPGGGGS
(SEQ ID NO. 15)
E5: GGSSGAPPPPGGGGS
(SEQ ID NO. 16)
E6: GGGSSGAPPPSGGGGSGGGGSGGGGS

Notes: (Xaa)x-(Pro)y-(Xaa)z, wherein Xaa is one or any combination of a plurality of G, A and S, x, z≧3, 26≧x+y+z≧14, 10≧y≧3 and 1≧y/(x+z)≧0.13. An N-terminal of the connecting peptide is connected with a C-terminal of the first region through a peptide bond, and a C-terminal of the connecting peptide is connected with an N-terminal of the HSA through a peptide bond.

Enhancement

| area | X | Y | Z | X + Y + Z | Y/X + Z |
|---|---|---|---|---|---|
| E1 | 5 | 3 | 6 | 14 | 0.272727 |
| E2 | 10 | 3 | 11 | 24 | 0.142857 |
| E3 | 4 | 10 | 7 | 21 | 0.909091 |
| E4 | 14 | 5 | 5 | 24 | 0.263158 |
| E5 | 6 | 4 | 5 | 15 | 0.363636 |
| E6 | 7 | 3 | 16 | 26 | 0.130435 |

2. Amplification of HSA Gene

Nucleotide coding sequence of HSA (SEQ ID NO. 7):

GATGCACACAAGAGTGAGGTTGCTCATCGGTTTAAAGATTTGGGAGAAGA
AAATTTCAAAGCCTTGGTGTTGATTGCCTTTGCTCAGTATCTTCAGCAGT
GTCCATTTGAAGATCATGTAAAATTAGTGAATGAAGTAACTGAATTTGCA
AAAACATGTGTTGCTGATGAGTCAGCTGAAAATTGTGACAAATCACTTCA
TACCCTTTTTGGAGACAAATTATGCACAGTTGCAACTCTTCGTGAAACCT
ATGGTGAAATGGCTGACTGCTGTGCAAAACAAGAACCTGAGAGAAATGAA
TGCTTCTTGCAACACAAAGATGACAACCCAAACCTCCCCCGATTGGTGAG
ACCAGAGGTTGATGTGATGTGCACTGCTTTTCATGACAATGAAGAGACAT
TTTTGAAAAAATACTTATATGAAATTGCCAGAAGACATCCTTACTTTTAT
GCCCCGGAACTCCTTTTCTTTGCTAAAAGGTATAAAGCTGCTTTTACAGA
ATGTTGCCAAGCTGCTGATAAAGCTGCCTGCCTGTTGCCAAAGCTCGATG
AACTTCGGGATGAAGGGAAGGCTTCGTCTGCCAAACAGAGACTCAAGTGT
GCCAGTCTCCAAAAATTTGGAGAAAGAGCTTTCAAAGCATGGGCAGTAGC
TCGCCTGAGCCAGAGATTTCCCAAAGCTGAGTTTGCAGAAGTTTCCAAGT
TAGTGACAGATCTTACCAAAGTCCACACGGAATGCTGCCATGGAGATCTG
CTTGAATGTGCTGATGACAGGGCGGACCTTGCCAAGTATATCTGTGAAAA
TCAAGATTCGATCTCCAGTAAACTGAAGGAATGCTGTGAAAAACCTCTGT
TGGAAAAATCCCACTGCATTGCCGAAGTGGAAAATGATGAGATGCCTGCT
GACTTGCCTTCATTAGCTGCTGATTTTGTTGAAAGTAAGGATGTTTGCAA
AAACTATGCTGAGGCAAAGGATGTCTTCCTGGGCATGTTTTTGTATGAAT
ATGCAAGAAGGCATCCTGATTACTCTGTCGTGCTGCTGCTGAGACTTGCC
AAGACATATGAAACCACTCTAGAGAAGTGCTGTGCCGCTGCAGATCCTCA
TGAATGCTATGCCAAAGTGTTCGATGAATTTAAACCTCTTGTGGAAGAGC
CTCAGAATTTAATCAAACAAAATTGTGAGCTTTTTGAGCAGCTTGGAGAG
TACAAATTCCAGAATGCGCTATTAGTTCGTTACACCAAGAAAGTACCCCA
AGTGTCAACTCCAACTCTTGTAGAGGTCTCAAGAAACCTAGGAAAAGTGG
GCAGCAAATGTTGTAAACATCCTGAAGCAAAAAGAATGCCCTGTGCAGAA
GACTATCTATCCGTGGTCCTGAACCAGTTATGTGTGTTGCATGAGAAAAC
GCCAGTAAGTGACAGAGTCACAAAATGCTGCACAGAGTCCTTGGTGAACA
GGCGACCATGCTTTTCAGCTCTGGAAGTCGATGAAACATACGTTCCCAAA
GAGTTTAATGCTGAAACATTCACCTTCCATGCAGATATATGCACACTTTC
TGAGAAGGAGAGACAAATCAAGAAACAAACTGCACTTGTTGAGCTTGTGA
AACACAAGCCCAAGGCAACAAAAGAGCAACTGAAAGCTGTTATGGATGAT
TTCGCAGCTTTTGTAGAGAAGTGCTGCAAGGCTGACGATAAGGAGACCTG
CTTTGCCGAGGAGGGTAAAAAACTTGTTGCTGCAAGTCAAGCTGCCTTAG
GCTTATAA

Primer Design:

GLP-1/P1 (SEQ ID NO. 26):
5′-TCTCTCGAGAAAAGACACGGCGAAGGGACCTTTACCAGTG-3′
(XhoI enzyme restriction site)
HSA/P1 (SEQ ID NO. 27):
5′-GATGCACACAAGAGTGAGG-3′
HSA/P2 (SEQ ID NO. 28):
5′-TTAGCGGCCGCTTATAAGCCTAAGGCAGCTTG-3′-(NotI
enzyme restriction site)

A Human Serum Albumin/HSA/ALB Gene cDNA Clone/ORF Clone gene (Sino Biological Inc.) was used as a template, HSA/P1 and HSA/P2 were used as primers, an HSA segment was amplified, and a PCR system included 0.5 μl of template, 1 μl of 25 μmol/L HSA/P1 and HSA/P2 respectively, 4 μl of 2 mmol/L dNTP, 10 μl of 5× PS reaction buffer solution, 2.5U of PrimerStar DNA polymerase and ddH₂O added to 50 μl.

PCR conditions included denaturation for 10 min at 98° C. and lmin 48 sec at 68° C., 25 cycles and then heat preservation at 4° C. For PCR products, bands with molecular weight of about 1750 bp were recovered through gel extraction by using agarose gel electrophoresis.

3. Amplification of Fusion Gene

3.1 Amplification of (GLP-1 Analogue)2-HSA Fusion Gene

Mixture of (GLP-1 analogue)2 gene segments and PCR products of HSA mixed by equal mole was used as a template, GLP-1/P1 and HSA/P2 were used as primers, (GLP-1 analogue)₂-HSA was amplified, and a PCR system included 0.5 μl of template, 1 μl of 25 μmol/L GLP-1/P1 and HSA/P2 respectively, 4 μl of 2 mmol/L dNTP, 10 μl of 5× PS reaction buffer solution, 2.5U of PrimerStar DNA polymerase and ddH₂O added to 50 μl. PCR conditions included 10 sec at 98° C. and 2 min 30 sec at 68° C., 25 cycles and then heat preservation at 4° C. For PCR products, bands with molecular weight of about 1950 bp were recovered through gel extraction by using agarose gel electrophoresis.

3.2 Amplification of GLP-1 Analogue-(Gly₄Ser)₃-HSA Fusion Gene

Mixture of GLP-1 analogue-(Gly₄Ser)₃ gene segments and PCR products of HSA mixed by equal mole was used as a template, a PCR system and PCR conditions were the same as 3.1, and for PCR products, bands with molecular weight of about 1930 bp were recovered through gel extraction by using agarose gel electrophoresis.

3.3 Amplification of GLP-1 Analogue-(Gly₄Ser)₄-HSA Fusion Gene

Mixture of GLP-1 analogue-(Gly₄Ser)₄ gene segments and PCR products of HSA mixed by equal mole was used as a template, a PCR system and PCR conditions were the same as 3.1, and for PCR products, bands with molecular weight of about 1950 bp were recovered through gel extraction by using agarose gel electrophoresis.

3.4 Amplification of GLP-1 Analogue-E1-HSA Fusion Gene

Mixture of GLP-1 analogue-E1 gene segments and PCR products of HSA mixed by equal mole was used as a template, a PCR system and PCR conditions were the same as 3.1, and for PCR products, bands with molecular weight of about 1930 bp were recovered through gel extraction by using agarose gel electrophoresis.

3.5 Amplification of GLP-1 Analogue-E2-HSA Fusion Gene

Mixture of GLP-1 analogue-E2 gene segments and PCR products of HSA mixed by equal mole was used as a template, a PCR system and PCR conditions were the same as 3.1, and for PCR products, bands with molecular weight of about 1960 bp were recovered through gel extraction by using agarose gel electrophoresis.

3.6 Amplification of GLP-1 Analogue-E3-HSA Fusion Gene

Mixture of GLP-1 analogue-E3 gene segments and PCR products of HSA mixed by equal mole was used as a template, a PCR system and PCR conditions were the same as 3.1, and for PCR products, bands with molecular weight of about 1940 bp were recovered through gel extraction by using agarose gel electrophoresis.

3.7 Amplification of GLP-1 Analogue-E4-HSA Fusion Gene

Mixture of GLP-1 analogue-E4 gene segments and PCR products of HSA mixed by equal mole was used as a template, a PCR system and PCR conditions were the same as 3.1, and for PCR products, bands with molecular weight of about 1960 bp were recovered through gel extraction by using agarose gel electrophoresis.

3.8 Amplification of GLP-1 Analogue-E5-HSA Fusion Gene

Mixture of GLP-1 analogue-E5 gene segments and PCR products of HSA mixed by equal mole was used as a template, a PCR system and PCR conditions were the same as 3.1, and for PCR products, bands with molecular weight of about 1930 bp were recovered through gel extraction by using agarose gel electrophoresis.

3.9 Amplification of GLP-1 Analogue-E6-HSA Fusion Gene

Mixture of GLP-1 analogue-E6 gene segments and PCR products of HSA mixed by equal mole was used as a template, a PCR system and PCR conditions were the same as 3.1, and for PCR products, bands with molecular weight of about 1970 bp were recovered through gel extraction by using agarose gel electrophoresis.

4. Construction of Fusion Protein Expression Plasmid

4.1 Construction of (GLP-1 Analogue)2-HSA Expression Plasmid

Firstly XhoI and NotI double enzyme restriction was performed to an expression vector plasmid pPIC9. Specific conditions were as follows: 10 μl of expression vector plasmid pPIC9; 1 μl of XhoI, 1 μl of NotI, and 4 μl of 10× enzyme restriction buffer solution (H) (purchased from Takara); and 24 μl of ddH2O and total volume of 400 Similar double enzyme restriction was performed to a (GLP-1 analogue)2-HSA segment. Reaction for 2 h in a 37° C. constant-temperature water bath was performed, and linearized plasmid DNA and (GLP-1 analogue)2-HSA gene segment were recovered through agarose gel electrophoresis. The recovered vector and gene segment were ligated to construct a fusion protein expression plasmid (GLP-1 analogue)2-HSA/pPIC9. A ligation system was generally 10 μl in volume, with the molar ratio of the vector to the gene segments being 1: (2-10), including 1 μl of 10xT4 DNA ligase buffer solution, 1 μl of T4 DNA ligase and sterile water added to 10 μl. Ligation reaction was performed for 1 h in a 16° C. constant-temperature water bath. Ligation products were transformed competent cells E. coli Top10 , transformed clone plaques were subjected to PCR identification by using general primers 5′ AOX1 and 3′ AOX1 as primers, correctly identified cloned bacteria solution was delivered to GenScript Corporation and sequencing was performed by using general primers 5′ AOX1 and 3′ AOX1. As verified by sequencing, the expectation was met.

4.2 Construction of GLP-1 Analogue-(Gly₄Ser)₃-HSA Expression Plasmid

Except the fusion protein gene segment which was replaced by a GLP-1 analogue-(Gly₄Ser)₃-HSA fusion gene, others were the same as 4.1, and as verified by sequencing, the expectation was met.

4.3 Construction of GLP-1 Analogue-(Gly4Ser)4-HSA Expression Plasmid

Except the fusion protein gene segment which was replaced by a GLP-1 analogue-(Gly₄Ser)₄-HSA fusion gene, others were the same as 4.1, and as verified by sequencing, the expectation was met.

4.4 Construction of GLP-1 Analogue-E1-HSA Expression Plasmid

Except the fusion protein gene segment which was replaced by a GLP-1 analogue-E1-HSA fusion gene, others were the same as 4.1, and as verified by sequencing, the expectation was met.

4.5 Construction of GLP-1 Analogue-E2-HSA Expression Plasmid

Except the fusion protein gene segment which was replaced by a GLP-1 analogue-E2-HSA fusion gene, others were the same as 4.1, and as verified by sequencing, the expectation was met.

4.6 Construction of GLP-1 Analogue-E3-HSA Expression Plasmid

Except the fusion protein gene segment which was replaced by a GLP-1 analogue-E3-HSA fusion gene, others were the same as 4.1, and as verified by sequencing, the expectation was met.

4.7 Construction of GLP-1 Analogue-E4-HSA Expression Plasmid

Except the fusion protein gene segment which was replaced by a GLP-1 analogue-E4-HSA fusion gene, others were the same as 4.1, and as verified by sequencing, the expectation was met.

4.8 Construction of GLP-1 Analogue-E5-HSA Expression Plasmid

Except the fusion protein gene segment which was replaced by a GLP-1 analogue-E5-HSA fusion gene, others were the same as 4.1, and as verified by sequencing, the expectation was met.

4.9 Construction of GLP-1 Analogue-E6-HSA Expression Plasmid

Except the fusion protein gene segment which was replaced by a GLP-1 analogue-E6-HSA fusion gene, others were the same as 4.1, and as verified by sequencing, the expectation was met.

### Embodiment 2: Construction of Engineering Bacteria for Fusion Protein Expression

Clones respectively containing (GLP-1 analogue)₂-HSA/pPIC9, GLP-1 analogue-(Gly₄Ser)₃-HSA/pPIC9, GLP-1 analogue-(Gly₄Ser)₄-HSA/pPIC9, GLP-1 analogue-E1-HSA/pPIC9, GLP-1 analogue-E2-HSA/pPIC9, GLP-1 analogue-E3-HSA/pPIC9, GLP-1 analogue-E4-HSA/pPIC9, GLP-1 analogue-E5-HSA/pPIC9 and GLP-1 analogue-E6-HSA/pPIC9 expression vector plasmids were selected .The expression vector plasmids were respectively extracted, then respectively linearized by using Sall. The linearized plasmid DNA were respectively recovered through agarose gel electrophoresis, and finally were respectively transformed to Pichia pastoris GS115 competent cells by using an electrotransformation method. After electric shock, 1 ml of 1M sorbitol solution was added to cell and mixed immediately, then the solution was transferred to a 1.5 ml centrifugal tube and placed at 30° C. for 1.5 h, then the cell suspension was coated on RDB selective plates with every 300 μl of cell suspension per. The plates were cultured at 30° C. for culture until single colonies occurred. The positive colonies were transferred to fresh RDB plates and cultured for 24 h, then single colonies, corresponding to each GLP-1 analogue fusion protein, which grown on the RDB plates were respectively selected and inoculated in 10 ml of BMGY culture medium, cultured for 24 h at 30° C. and 250 rpm. Cell suspension was placed and the supernatant was discarded, then the cells were resuspended by using 10 ml of BMMY (2% methanol). Cells were induced for 48 h at 30° C. and 250 rpm, then the supernatant was collected by centrifugation to detect the expression of the fusion proteins through 10% SDS-PAGE electrophoresis. The N-terminals of the fusion proteins were sequenced if the size of electrophoresis bands met the expectation, and the sequencing results which met the expectation means engineering strains with each GLP-1 analogue fusion protein were construced successfully.

Specific conditions for linearizing plasmids were as follows: 60 μl of expression vector plasmid, 2.5 μl of SalI, 20 μl of 10× buffer solutions (H) and added to 200 μl by ddH₂O. Reaction was performed for 3 h in a 37° C. constant-temperature water bath.

A specific method for preparing competent cells comprised the following steps: firstly preparing colonies, selecting yeast single colonies, inoculating the single colonies in a 50 ml triangular flask containing 5 ml of YPD culture medium, and performing culture at 30° C. and 250 rpm overnight; then taking and inoculating 30 μl of culture into a 250 ml triangular flask containing 50 ml of YPD culture medium, and performing culture at 30° C. and 250 rpm overnight until OD600 reached 1-1.5; precooling cell culture on ice for 10 min, then performing centrifugation for 5 min at 4° C. and 1500×g, discarding supernatant, and resuspending the precipitation of cells with 40 ml of precooled sterile water, centrifugating, then resuspending the precipitation of cells with 25 ml of precooled sterile water, recentrifugating and resuspending the precipitation of cells with 5 ml of precooled 1M sorbitol solution, then recentrifugating and resuspending the precipitation of cells with 80 μl of precooled 1M sorbitol solution.

A specific electrotransformation method comprised the following steps: uniformly mixing 10 μl of linearized plasmids with 80 μl of the competent cells, transferring the mixture to a 0.2 cm ice-precooled electrotransformation cup, placing the electrotransformation cup in an ice bath for 5 min and then performing electric shock by using 1500V voltage.

### Embodiment 3: Preparation of GLP-1 Analogue Fusion Proteins

Referring to Manual of Methods for Expression of Recombinant Proteins in Pichia pastoris (Invitrogen Corporation), strains, expressing each GLP-1 analogue fusion protein, which were obtained in embodiment 2 were inoculated in YPD culture medium. Culture was performed by shaking at 30° C. and 220-280 rpm until the wet weight of the cells reached about 50 g/L, the cells were inoculated into bioreaction (Biostat C10, Sartorius) by a dosage of 10%. Culture was performed for 20 h at 30° C., pH 5.0 and 30% of dissolved oxygen saturation. Then methanol was continuously fed to start induction. The dissolved oxygen saturation was controlled at 40%. The temperature was reduced to 22° C. after induction for 4 h. The induction was ended after 50 h and the supernatant was collected by centrifugation for 15 min at 10000×g and fermented supernatant was collected.

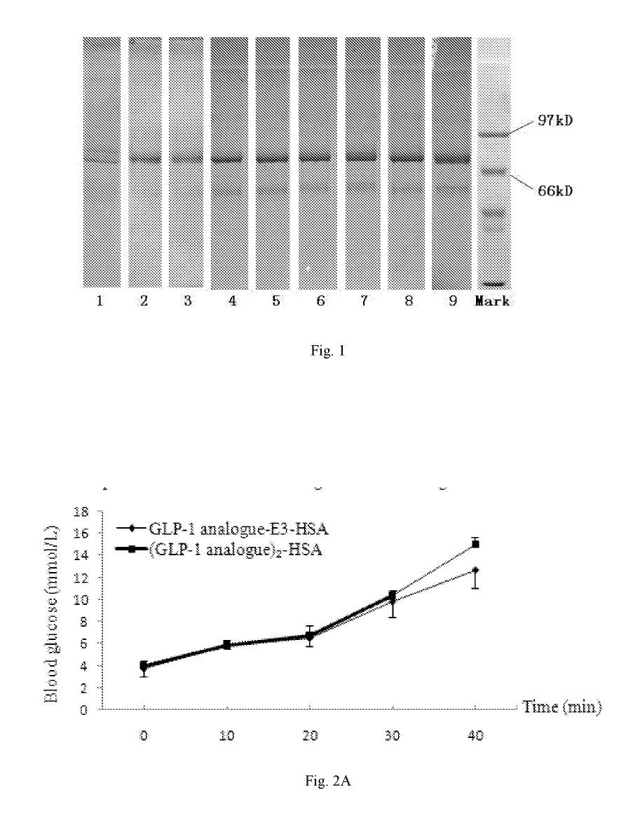

BLUE affinity, PHE hydrophobic, DEAE ion exchange and gel exclusion four-step chromatography was adopted for purification. Firstly, the fermented supernatant was diluted by three times by using 20 mM pH 7.0 sodium phosphate solution, then the solution passed through a Blue Sepharose Fast Flow (XK 50/20, GE healthcare) affinity chromatography column, balancing was performed by using PBS, and then the target protein was eluted by using 2M NaCl and 20 mM pH 6.5 sodium phosphate solution. (NH₄)₂SO₄ was added into the collected protein solution to enable the final concentration to reach 0.5M, the protein solution passed through a PHE Sepharose Fast Flow (XK 50/20, GE healthcare) chromatography column, balancing was performed by using 0.6M (NH₄)₂SO₄, and then the protein was eluted by using 5 mM pH 6.5 sodium phosphate buffer solution. The collected protein was diluted by two times by using 5 mM pH 6.5 sodium phosphate buffer solution, then the solution passed through an ion exchange chromatography column, and the target protein was eluted directly by using PBS by adopting a DEAE Sepharose Fast Flow (XK 50/20, GE healthcare) chromatography column. Finally, desalination was performed through a Sephadex G25 coarse (XK 50/60, GE healthcare) gel chromatography column to realize displacement into 5 mM pH 6.5 sodium phosphate buffer solution. The expression supernatant and the purified fusion protein were respectively analyzed by using non-reductive SDS-PAGE. As shown in FIG. 1, there was a great difference in stability of GLP-1 analogue fusion proteins with different structures during expression, wherein the stability of (GLP-1 analogue)2-HSA is the poorest.

### Embodiment 4: In-Vitro Activity Test

According to the literature (Zlokamik G, Negulescu P A, Knapp T E, Mere L, Burres N, Feng L, Whitney M, Roemer K, Tsien R Y. Science. 279 (5347): 84-8. (1998)), HEK-293 cells carrying with human GLP-1 receptors and CRE-Luc reporter genes were constructed, and DMEM culture containing 10% of FBS according to 50000 cells/well/200 μl was used for inoculation into a Costar 96-well cell culture plate. On the second day after inoculation, culture solution was absorbed away, 50 μl of serum-free DMEM culture solution of stepwise diluted GLP-1 analogue fusion proteins containing 500 μM IBMX was added into each well, incubation was performed for 5-6 h, then 50 μl of luciferase substrate (Bright-Glo™ Luciferase Assay System, Promega, E2620) was added.Reaction was performed for 2 min, then the solution was transferred to a Costar 96-well all-white micro-well plate. Fluorescence values were determined on a multifunctional ELISA microplate reader (SpectraMax M5 system, Molecular Device). A dose-response curve was depicted according to the fluorescence values and an EC₅₀ value was determined. By taking the activity of (GLP-1 analogue)₂-HSA as 100%, relative activity of each fusion protein was calculated. Results were as shown in Table 1. The in-vitro activity of Gly₄Ser as a connecting peptide was substantially similar to that of a GLP-1 analogues as a connecting peptide; and however, when a segment of sequences (E1-E6) according to claim 1 was inserted between the GLP-1 analogue and HSA, the in-vitro activity of the fusion protein was improved by about 7-10 times.

TABLE 1

| | | Relative | Standard |
|---|---|---|---|
| | Fusion protein | activity (%) | deviation |
| 1 | (GLP-1 analogue)₂-HSA | 100 | 17 |
| 2 | GLP-1 analogue-(Gly₄Ser)₃-HSA | 103 | 18 |
| 3 | GLP-1 analogue-(Gly₄Ser)₄-HSA | 108 | 22 |
| 4 | GLP-1 analogue-E1-HSA | 752 | 98 |
| 5 | GLP-1 analogue-E2-HSA | 823 | 124 |
| 6 | GLP-1 analogue-E3-HSA | 1108 | 89 |
| 7 | GLP-1 analogue-E4-HSA | 957 | 141 |
| 8 | GLP-1 analogue-E5-HSA | 1003 | 125 |
| 9 | GLP-1 analogue-E6-HSA | 763 | 99 |

### Embodiment 5: In-Vitro Stability Analysis

High-purity GLP-1 analogue fusion protein stock solution was taken, proper amounts of sodium chloride, disodium hydrogen phosphate and sodium dihydrogen phosphate were added, pH was regulated to 7.4 by using sodium hydroxide or hydrochloric acid, and then water for injection was added to enable lml of solution to contain 5.0 mg of GLP-1 analogue protein, 9 mg of sodium chloride and 20 μmol of phosphate. Bacteria were removed by using a 0.22 μm PVDF or PES filter membrane. The solution was aseptically packaged in a penicillin bottle under a class-100 environment. The sample was stored in a stability test box at 25° C., and samples were respectively taken at the 0^{th}, 1^{st} and 3^{rd} month and was stored in a −70° C. refrigerator for detection. All samples to be analyzed were combined and SDS-PAGE purity and cell biological activity were detected. The method for detecting the SDS-PAGE purity was as described in embodiment 1 and the loading amount of the sample to be detected was 10 ug. In addition, 1 ug, 0.5 ug, 0.2 ug, 0.1 ug and 0.05 ug of self-control were loaded. Optical density scanning was performed to obtain a standard curve, the percentage content of each impure protein was calculated and finally the purity of the fusion protein was calculated. The method for determining in-vitro activity was as described in embodiment 4, and the activity of each sample at the zero month was taken as 100%. Before activity determination, the sample was separated by using a Superdex 75 10/30 molecular sieve column (GE Healthcare) to remove degraded segments with molecular weight which was smaller than 10000 Da. So as to avoid the disturbance thereof to the activity determination. Results were as shown in Table 2. When the GLP-1 analogue was inserted as a connecting peptide between the GLP-analogue and HSA, the activity preservation rate was the poorest and the fusion protein was the most instable.

TABLE 2

| | | | | | Activity | | |
|---|---|---|---|---|---|---|---|
| | | SDS-PAGE purity (%) | | | preservation rate (%) | | |
| | Fusion protein | 0 | 1 | 3 | 0 | 1 | 3 |
| 1 | (GLP-1 analogue)₂-HSA | 97.1 | 65.3 | 34.3 | 100 | 45.4 | 13.5 |
| 2 | GLP-1 analogue-(Gly₄Ser)₃-HSA | 97.3 | 84.3 | 67.3 | 100 | 79.0 | 47.2 |
| 3 | GLP-1 analogue-(Gly₄Ser)₄-HSA | 98.0 | 89.4 | 66.0 | 100 | 85.5 | 45.3 |
| 4 | GLP-1 analogue-E1-HSA | 97.5 | 93.4 | 77.9 | 100 | 97.6 | 60.6 |
| 5 | GLP-1 analogue-E2-HSA | 98.3 | 94.3 | 72.8 | 100 | 93.4 | 57.9 |
| 6 | GLP-1 analogue-E3-HSA | 97.9 | 95.1 | 77.3 | 100 | 91.7 | 49.8 |
| 7 | GLP-1 analogue-E4-HSA | 97.5 | 94.8 | 75.2 | 100 | 96.2 | 55.6 |
| 8 | GLP-1 analogue-E5-HSA | 98.4 | 95.7 | 81.2 | 100 | 93.3 | 47.2 |
| 9 | GLP-1 analogue-E6-HSA | 98.2 | 96.7 | 80.1 | 100 | 91.7 | 53.9 |

### Embodiment 6: Serum Stability Analysis

Purified high-purity GLP-1 analogue fusion protein stock solution was taken and added into monkey serum according to a volume ratio of 1:25, filtration was performed to remove bacteria, the solution was aseptically packaged in a penicillin bottle and incubation was performed at 37° C. Samples were taken at the 0^{th}, 15^{th} and 30^{th} day and stored in a −70° C. refrigerator for detection. All samples to be analyzed were combined, and fusion protein concentration was determined through a sandwich ELISA method by using Anti-GLP-1 monoclonal antibodies (Antibodyshop) as capture antibodies and Goat anti-Human Albumin-HRP (Bethyl Laboratories) as detection antibodies. Since the capture antibodies were bound to the portion of the GLP-1 analogue of the fusion protein and the detection antibodies were bound to the portion of the albumin, the determined fusion protein concentration was positively correlated with the content of the undegraded portion. Results were as shown in Table 3. After 30 days, most (GLP-1 analogue)2-HSA in the monkey serum had already been degraded and about 40% of other samples were reserved.

TABLE 3
Change situation of content of fusion protein in serum with time

| | | Fusion protein content (%) | | |
|---|---|---|---|---|
| | Fusion protein | 0^{th} day | 15^{th} day | 30^{th} day |
| 1 | (GLP-1 analogue)₂-HSA | 100 | 37.4 | 17.8 |
| 2 | GLP-1 analogue-(Gly₄Ser)₃-HSA | 100 | 65.4 | 34.3 |
| 3 | GLP-1 analogue-(Gly₄Ser)₄-HSA | 100 | 69.3 | 37.6 |
| 4 | GLP-1 analogue-E1-HSA | 100 | 74.5 | 44.9 |
| 5 | GLP-1 analogue-E2-HSA | 100 | 72.0 | 41.2 |
| 6 | GLP-1 analogue-E3-HSA | 100 | 66.3 | 45.5 |
| 7 | GLP-1 analogue-E4-HSA | 100 | 73.2 | 48.2 |
| 8 | GLP-1 analogue-E5-HSA | 100 | 75.4 | 55.3 |
| 9 | GLP-1 analogue-E6-HSA | 100 | 76.9 | 45.1 |

Note:
the concentration determined on the 0^{th} day was taken as 100%.

### Embodiment 7: Mouse Intraperitoneal Glucose Tolerance Test

Totally 32 KM mice including 16 female mice and 16 male mice were taken and fed no food but water only overnight for 18 h, and then subcutaneous injection of 1.0 mg/kg HSA (control group), (GLP-1 analogue)₂-HSA, GLP-1 analogue-E3-HSA and GLP-1 analogue-E6-HSA was performed. After 1 hour and 8 hours after administration, intraperitoneal glucose tolerance tests (IPGTT) were respectively performed, intraperitoneal injection of 1.5 g/kg glucose was performed, and blood was taken before (t=0) glucose injection and after 10 min, 20 min, 30 min, 60 min, 90 min and 120 min after glucose injection to determine the content of glucose in blood (YSI2700 biochemical analyzer). Compared with the control group (HSA group), the blood glucose of the mice of the (GLP-1 analogue)2-HSA, GLP-1 analogue-E3-HSA and GLP-1 analogue-E6-HSA groups was obviously reduced, and the area under curve (AUC_{0-120 min}) of blood glucose was obviously smaller than that of the control group (results were as shown in Table 4). When the IPGTT was performed after 1 hour after administration, the blood glucose levels at respective time point among the three groups were similar, the areas under curve (AUC_{0-120 min}) of blood glucose were also similar, and no remarkable difference (P>0.05) existed among the groups; and however, when the IPGTT was performed after 8 hours after administration, the blood glucose levels of the mice of the GLP-1 analogue-E3-HSA and GLP-1 analogue-E6-HSA groups at 10-30 min were obviously lower than that of the (GLP-1 analogue)₂-HSA group, and the AUC_{0-120 min} of blood glucose was also obviously lower than that of the (GLP-1 analogue)2-HSA group (P<0.01). The results shown that both (GLP-1 analogue)₂-HSA and GLP-1 analogue-E3-HSA could effectively reduce mice fasting blood-glucose and had a long-acting feature, but compared with the (GLP-1 analogue)₂-HSA group, the GLP-1 analogue-E3-HSA and GLP-1 analogue-E6-HSA groups had more remarkable and continuous blood glucose reducing effects.

TABLE 4
Areas under curve (AUC_{0-120 min}) of blood glucose during IPGTT
at 1 h and 8 h after 2 single-dose administration to KM mice

| | | Animal | | | | | | | | | | | |
|---|---|---|---|---|---|---|---|---|---|---|---|---|---|
| Time | group | 1 | 2 | 3 | 4 | 5 | 6 | 7 | 8 | 9 | 10 | Mean | SEM |
| 1 h | HSA | 818 | 713 | 724 | 778 | 817 | 1028 | 1220 | 688 | 1005 | 1096 | 889 | 184 |
| | (GLP-1 analogue)₂-HSA | 544 | 692 | 679 | 640 | 528 | 727 | 589 | 763 | 671 | 649 | 648 | 76 |
| | GLP-1 analogue-E3-HSA | 775 | 645 | 596 | 501 | 563 | 520 | 690 | 688 | 553 | 643 | 617 | 86 |
| | GLP-1 analogue-E6-HSA | 654 | 731 | 638 | 602 | 554 | 498 | 512 | 620 | 578 | 621 | 601 | 69 |
| 8 h | HSA | 735 | 746 | 772 | 831 | 883 | 717 | 882 | 933 | 859 | 854 | 821 | 74 |
| | (GLP-1 analogue)₂-HSA | 691 | 585 | 762 | 570 | 580 | 656 | 705 | 430 | 496 | 642 | 612 | 100 |
| | GLP-1 analogue-E3-HSA | 447 | 528 | 525 | 469 | 562 | 348 | 515 | 624 | 527 | 358 | 490 | 87 |
| | GLP-1 analogue-E6-HSA | 502 | 485 | 445 | 412 | 450 | 520 | 471 | 465 | 542 | 399 | 469 | 45 |

### Embodiment 8: Pharmacodynamic Test of GLP-1 Analogue Fusion Protein After Single-Dose Subcutaneous Injection to Normal Rhesus Monkey

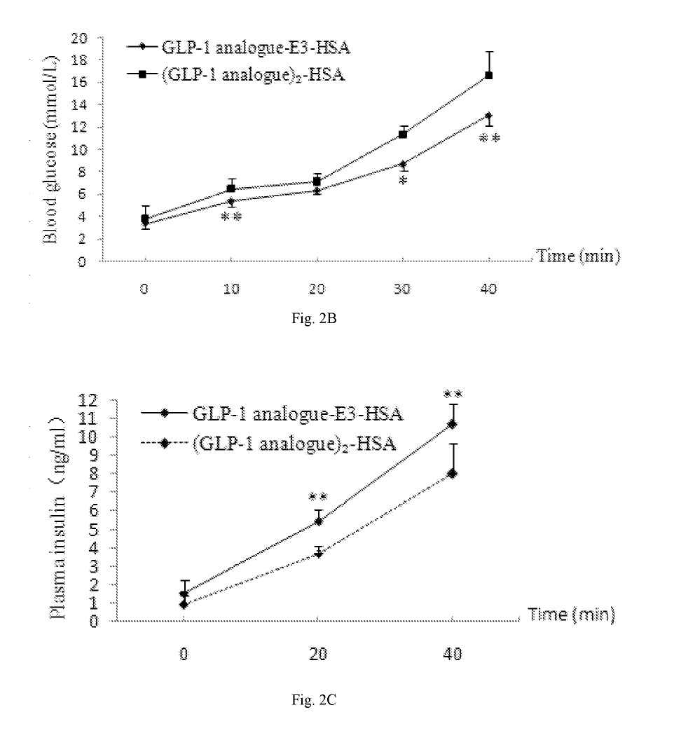

Single-dose subcutaneous injection of 0.5 mg/kg (GLP-1 analogue)₂-HSA or GLP-1 analogue-E3-HSA was performed to a rhesus monkey, stepwise intravenous glucose tests were performed after 24 h and 96 h, intravenous injection of glucose solution (20% dextrose solution, 200 mg/ml) was performed continuously for 20 min according to 10 mg/kg/min (3.0 ml/kg/h), and then glucose solution was administrated continuously for 20 min according to 25 mg/kg/min (7.5 ml/kg/h). Blood was acquired after 0, 10 min, 20 min, 30 min and 40 min after glucose injection to determine blood glucose and insulin. YS12700 biochemical analyzer was used for determining blood glucose and enzyme-linked immunosorbent assay (Insulin ELISA kit, DRG International, Inc.) was used for determining insulin. There was no remarkable difference in blood glucose between the two groups at respective time point after 1 d after administration (results were shown in FIGS. 2A-D). There was a remarkable difference (P<0.05 or P<0.01) between the groups at 10 min, 30 min and 40 min after 4 d after administration; and there was a remarkable difference (P<0.01) in insulin between the groups at time points 20 min and 40 min after 1 d and 4 d after administration. The results shown that, compared with (GLP-1 analogue)₂-HSA, GLP-1 analogue-E3-HSA could better promote the secretion of insulin and reduce the blood glucose level in the stepwise intravenous glucose test carried out to the normal rhesus monkey.

### Embodiment 9: Pharmacokinetic Research After Single-Dose Administration to Crab-Eating Macaque

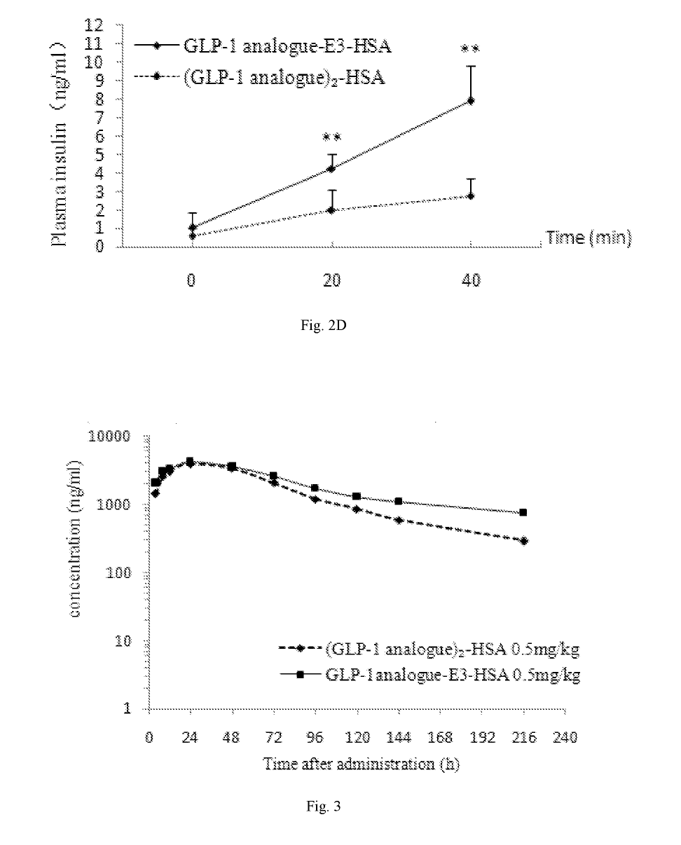

Single-dose subcutaneous injection of 0.5 mg/kg (GLP-1 analogue)₂-HSA and GLP-1 analogue E3-HSA was respectively performed to crab-eating macaques, blood was respectively acquired before administration (t=0) and after 4 h, 8 h, 12 h, 24 h, 48 h, 72 h, 96 h, 120 h, 144 h and 216 h after administration, serum was separated, cryopreservation was performed at −80° C. and then the serum was combined and detected. Concentration of fusion protein in the serum was determined by using Anti-GLP-1 monoclonal antibodies (Antibodyshop) as capture antibodies and Goat anti-Human Albumin-HRP (Bethyl Laboratories) as detection antibodies (see FIG. 3), and pharmacokinetic parameters (see Table 5) were calculated. The research shown that the half-life period of the 0.5 mg/kg GLP-1 analogue-E3-HSA in the body of the crab-eating macaque was 102 h (about 4 d) and the half-life period of the (GLP-1 analogue)₂-HSA was 60 h (2.5 d).

TABLE 5

| Parameters | (GLP-1 analogue)₂-HSA | GLP-1 analogue-E3-HSA |
|---|---|---|
| Cₘₐₓ(ng/ml) | 3954 | 4452 |
| Tₘₐₓ(h) | 24 | 24 |
| AUC_{0-∞}(ng/ml*h) | 356210 | 516613 |
| T_{1/2}(h) | 60 | 102 |
| CL(ml/h/kg) | 1.404 | 0.968 |

### Embodiment 10: Immunogenicity After Repetitive Subcutaneous Administration to Crab-Eating Macaque

Subcutaneous injection of 1 mg/kg (GLP-1 analogue)2-HSA and GLP-1 analogue-E3-HSA was weekly performed to crab-eating macaques, and administration was continuously performed for 3 months. Blood was respectively acquired before administration (t=0) and after 1 month, 2 months and 3 months after administration, serum was separated, cryopreservation was performed at −80° C. and then the serum was combined and detected. Monkey-anti-fusion protein antibodies which were possibly produced were determined by using enzyme-linked immunosorbent assay (ELISA). Corresponding fusion proteins were used as encrusting substances, to-be-detected serum samples of different dilution were added, and the titer of the antibodies was determined by using mouse-anti-monkey IgG as detection antibodies. Simultaneously, under similar determination conditions, human serum albumin was added as antagonist into the to-be-detected serum samples (final concentration of 60 μM) to further analyze the produced antibody specificity (see Table 6). Research results shown that, after repetitive administration, antibodies were produced by the both, the highest titer reached 1:6400, and the trends and titers of the antibodies produced by the both were substantially consistent. HSA was further added into serum for antagonistic analysis, results shown that the titer of the serum was obviously decreased under the existence of HSA and it indicated that the produced antibodies were substantially antagonized by HSA. Therefore, it indicated that most antibodies produced after repetitive injection of fusion protein to macaques were directed at the portion of HSA in the fusion protein and no anti-GLP-1 analogue antibodies were produced.

TABLE 6

| | | Titer of anti-fusion protein antibody | | | | Titer of anti-GLP-1 analogue antibody | | | |
|---|---|---|---|---|---|---|---|---|---|
| | | Before | | | | Before | | | |
| | Animal | admin- | 1 | 2 | 3 | admin- | 1 | 2 | 3 |
| Fusion protein | No. | istration | month | months | months | istration | month | months | months |
| (GLP-1 analogue)₂-HSA | 1 | N.D. | N.D. | 1:1600 | 1:6400 | N.D. | N.D. | N.D. | 1:100 |
| | 2 | N.D. | 1:100 | 1:6400 | 1:6400 | N.D. | N.D. | N.D. | 1:200 |
| | 3 | N.D. | N.D. | 1:1600 | 1:1600 | N.D. | N.D. | N.D. | N.D. |
| GLP-1 analogue-E3-HSA | 4 | N.D. | N.D. | N.D. | 1:100 | N.D. | N.D. | N.D. | N.D. |
| | 5 | N.D. | 1:100 | 1:6400 | 1:6400 | N.D. | N.D. | 1:100 | N.D. |
| | 6 | N.D. | N.D. | 1:1600 | 1:1600 | N.D. | N.D. | N.D. | N.D. |

Note:
antibody titer < 1:100 was defined as not detected (N.D.).

The above-mentioned embodiments are only used for exemplarily describing the principle and efficacies of the present invention instead of limiting the present invention. One skilled in the art can make modifications or changes to the above-mentioned embodiments without departing from the spirit and the range of the present invention. Therefore, all equivalent modifications or changes made by one who has common knowledge in the art without departing from the spirit and technical concept of the present invention shall be still covered by the claims of the present invention.

1. A GLP-1 analogue fusion protein, characterized in that a structure of the fusion protein is GLP-1 analogue-linker peptide-human serum albumin, the length of the linker peptide does not exceed 26 amino acids and a general formula is (Xaa)x-(Pro)y-(Xaa)z, wherein Xaa is one or any combination of a plurality of A and S, x, y and z are integers, x, z≧3, 26≧x+y+z≧14, 10≧y≧3, 1≧y/(x+z)≧0.13, an N-terminal of the linker peptide is connected with a C-terminal of the GLP-1 analogue through a peptide bond, and a C-terminal of the linker peptide is connected with an N-terminal of the human serum albumin through a peptide bond.

2. The GLP-1 analogue fusion protein according to claim 1, characterized in that the GLP-1 analogue is any one of follows:
a) having an amino acid sequence of SEQ ID NO. 1;
b) having an amino acid sequence which maintains 85%, preferably 90%, more preferably 95% or more preferably 99% of homology with SEQ ID NO. 1;
c) comprising 2 or 3 repetitive sequences of the GLP-1 analogue of a) or b), or comprising 2 or 3 repetitive sequences of a GLP-1; and
d) being Exendin-4.

3. The GLP-1 analogue fusion protein according to claim 1, characterized in that an amino acid sequence of the linker peptide is any one of SEQ ID NO. 11-16.

4. The GLP-1 analogue fusion protein according to claim 1, characterized in that an amino acid sequence of the human serum albumin is SEQ ID NO. 2 or at least maintains 85%, preferably 90%, more preferably 95% or more preferably 99% of homology with SEQ ID NO. 2.

5. The GLP-1 analogue fusion protein according to claim 1, characterized in that an amino acid sequence of the GLP-1 analogue fusion protein is selected from SEQ ID NO. 3-5.

6. A polynucleotide coding the GLP-1 analogue fusion protein according to claim 1.

7. The polynucleotide according to claim 6, characterized in that a sequence of the polynucleotide is selected from SEQ ID NO. 8-10.

8. A method for preparing the GLP-1 analogue fusion protein according to claim 1, the method comprising the following steps: constructing an expression vector containing a gene sequence of the GLP-1 analogue fusion protein, then transforming the expression vector to a host cell for induced expression, and separating and obtaining the fusion protein from expression products.

9. The method for preparing the GLP-1 analogue fusion protein according to claim 8, characterized in that the expression vector is pPIC9; and the host cell is Pichia pastoris.

10. The method for preparing the GLP-1 analogue fusion protein according to claim 8, characterized in that a method for separating and obtaining the fusion protein from the expression products comprises the step of separating and obtaining the fusion protein by adopting a three-step chromatographic method which joints affinity chromatography, hydrophobic chromatography and ion exchange chromatography.

11. Application of the GLP-1 analogue fusion protein according to claim 1 to preparation of medicines for treating diabetes and related diseases.

12. A pharmaceutical composition for treating diabetes and diabetes-related diseases, containing the GLP-1 analogue fusion protein according to claim 1 and at least one pharmaceutically acceptable carrier or excipient.

13. A method for treating diabetes and diabetes-related diseases, comprising the step of administrating the GLP-1 analogue fusion protein according to claim 1 to an object.
